(19) United States
(12) Patent Application Publication
(10) Pub. No.: US 2014/0275864 A1
(43) Pub. Date: Sep. 18, 2014

(54) Polyurethane dressing and method for making same

(71) Applicant: Thomas J. Drury, Tolland, CT (US)

(72) Inventor: Thomas J. Drury, Tolland, CT (US)

(73) Assignee: HYDROFERA, LLC

(21) Appl. No.: 13/815,717

(22) Filed: Mar. 15, 2013

(51) Int. Cl. A61B 5/145; A61L 15/26; A61F 13/02; A61L 15/46

(52) U.S. Cl. CPC A61B 5/14546; A61L 15/46; A61L 15/26; A61F 13/0289
USPC 600/309; 427/2.31

(57) ABSTRACT

The present invention is directed toward a wound dressing comprising a foam body made of polyurethane material treated with a plurality of colored biocidal dyes with at least one of the dyes being gram positive and at least one of the dyes being gram negative to dye the body a distinct color. The body has at least one planar surface and ranges from about 1 mm to about 10 mm in thickness and has an outer film cover and border with a hydrogel adhesive secured to the body. The foam body changes color when placed in contact with an infected wound site.

### RELATED APPLICATIONS

This is a continuation-in-part application from U.S. patent application Ser. No. 13/507,581 filed Jul. 12, 2012.

### STATEMENT REGARDING FEDERALLY SPONSORED RESEARCH OR DEVELOPMENT

None.

### REFERENCE TO SEQUENCE LISTING, A TABLE OR A COMPUTER PROGRAM LISTING COMPACT DISC APPENDIX

None.

### BACKGROUND OF THE INVENTION

1. Field of Invention

The present invention relates generally to a wound care dressing and more specifically relates to a bacteriostatic repositionable wound care dressing constructed of a synthetic polymer foam body preferably of polyurethane having a combination biocidal dye with non-oxidant gram-positive and gram-negative characteristics, preferably Methylene Blue and Crystal Violet biocidal dyes bound to the foam. The foam body is secured to an adhesive cover and border made from a plastic film. The foam body ranges from about 1 mm to about 10 mm in thickness and when placed on an open wound, color changes to indicate infection at the wound site.

2. Background of the Invention

There are a number of major problems encountered in present day wound dressings. Such wound dressings are primarily of the cellulose type and the sponge type. Cellulose dressings have probably been used in some form since the beginning of recorded history and are generally constructed of cellulose materials such as cotton. These wound dressings leave linting fibers, allow bacterial growth adjacent the wound and allow wound growth extension into the fibers of the dressing. Removal of the dressing also causes tearing of the wound when the dressing is removed and can leave fibrous fragments in the wound. Because of the fiber spacing in such dressings they also do not present a barrier against direct exposure to air and organisms carried in the air. These wound dressings also do not provide a color change allowing a nurse or technician to observe infection at the wound site.

Advances in the development of synthetic polymers have produced numerous changes in wound care dressings resulting in polymeric foams, polymeric films, particulate and fibrous polymers, hydrogels and hydrocolloids. These developments have resulted in sponge type dressings being widely used but unfortunately these dressings also suffer from wound growth into the cells of the sponges, failure to kill bacteria and other infectious agents, lack of absorption and wound tearing problems which occur when the dressings are removed.

Polyvinyl acetal and polyurethane sponges have been used in the treatment of wounds for many years and have an excellent safety record. Both polymers absorb liquid into the sponge where all bacteriostatic activity takes place.

U.S. Pat. No. 4,098,728 issued Jul. 4, 1978 discloses the use of polyvinyl acetal material having a fast wicking and high liquid holding capacity for medical usage.

U.S. Pat. No. 5,071,648, issued on Dec. 10, 1991 discloses a polyvinyl acetal material with a complex of iodine which forms a sponge releasing controlled amounts of iodine sufficient to kill germ cells with minimum toxicity to the surrounding tissue. Likewise, U.S. Pat. No. 5,744,150 issued on Apr. 28, 1998 and U.S. Pat. No. 5,928,665 issued Jul. 27, 1999 disclose a method for producing an antimicrobial iodine polyvinyl acetal sponge which is soaked in an aqueous bath of 20% to 70% triethylene glycol. The resultant product is a wound dressing including an iodine complexed polyvinyl acetal sponge material in which alkylene glycol is applied to the surface of the sponge to soften the sponge and impart a yellow-gold coloration onto the outer surface of the sponge indicating the activation of the antimicrobial elements complexed in the sponge material.

U.S. Pat. No. 5,810,755 issued Sep. 22, 1998 discloses a medicated wound dressing with an open cell foam polymeric compound of polyvinyl alcohol complexed with elemental iodine. None of these references exhibit a color change of the wound dressing indicating infection at the wound site.

U.S. Pat. No. 5,554,659 issued Sep. 10, 1996 and U.S. Pat. No. 5,556,391 issued Sep. 17, 1996 are directed toward a molded porous polyvinyl alcohol sponge including an outer skin having an average pore size smaller than the interior portion of the product capable of absorbing and passing water to the interior portions of the sponge.

U.S. Pat. No. 5,811,471, issued Sep. 22, 1998 discloses a polyvinyl acetal polymer sponge which has a germicidal disinfectant dye bound thereto which is used as a tampon while U.S. Pat. No. 5,447,505 issued Sep. 5, 1995 discloses the use of polyvinyl acetal sponge surgical dressing. U.S. Pat. No. 6,183,764 issued Feb. 6, 2011 and U.S. Pat. No. 6,361,786 issued Mar. 26, 2002 are also directed toward a polyvinyl acetal polymer material which have a plurality of organic dyes absorbed therein to provide microbiocidal properties.

U.S. Pat. No. 6,613,347 issued Sep. 2, 2003 is directed toward a polyvinyl acetal sponge with a low durometer silicone smooth outer skin having less porosity then the foam center. The composite wound dressing allows moisture adsorption through the skin into the PVA sponge body but presents an outer surface precluding wound growth into the sponge material.

One problem with polyvinyl acetal sponge material is that it should be hydrated before use to soften the sponge.

None of the aforementioned prior art references are directed toward repositionable bacteriostatic gamma irradiated or electron beam sterilized wound dressings which exhibit color change when in contact with the wound to indicate if the site is infected. Bacteriostatic dressings better manage bioburden which helps the body's own immune system restore bacterial balance. The present inventive wound dressing changes its base color to white as an indicator that the wound site is infected and sustains bacteriostatic activity for up to 7 days.

### SUMMARY OF THE INVENTION

The present invention is directed toward a sterilized polyurethane foam wound care sponge dressing having a thickness ranging from about 1 mm to 10 mm with a cover and border made of a plastic film with a hydrogel adhesive which allows the dressing to be repositionable. The polyurethane sponge is washed and prepped with alcohol to open up the binding sites and then placed into a dye bath containing a gram-negative biocidal Methylene Blue dye and a gram-positive Crystal Violet dye which bind to the open sites of the polyurethane foam material. The sponge foam is rinsed until the unbound dye is removed from the sponge body and dried. An outer adhesive cover and boundary plastic skin is secured to the outer surface of the sponge. The composite wound dressing allows moisture adsorption into the polyurethane sponge body causing the dressing to expand preventing wound leakage and turns to white from its original blue color indicating infection at the wound site.

It is an object of the invention to provide a wound care dressing having an outer adhesive surface of different material from the body of the dressing allowing the dressing to be repositioned on a patient's body.

It is also an object of the invention to provide a wound care dressing where all germicidal activity takes place within the structure of the foam sponge.

It is yet another object of the invention to provide a wound care dressing which will not show a zone of inhibition beyond the contact area of the sponge.

It is still another object of the invention to provide a wound dressing which allows comfortable easy application of the dressing to the wound without hydration and repositioning of the dressing on the wound with minimal tearing of the wound area.

It is another object of the invention to provide a wound dressing which applies a biocidal material covering a full bacteria spectrum at a wound site.

It is yet another object of the invention to provide a wound dressing which exhibits a color change when the wound site is infected.

In the accompanying drawings, there is shown an illustrative embodiment of the invention from which these and other objectives, novel features and advantages will be readily apparent.

### BRIEF DESCRIPTION OF THE DRAWINGS

FIG. 1 is a plan view of the inventive wound care dressing;

FIG. 2 is an enlarged cross sectional view of an inventive wound care dressing shown in FIG. 1 taken along lines 2′-2;

FIG. 3 is a perspective view of the inventive wound care dressing placed on a patient showing color change; and

FIG. 4 is a block diagram of the process for making the polyurethane bound dye sponge dressing.

These and other objects, advantages, and novel features of the present invention will become apparent when considered with the teachings contained in the detailed disclosure along with the accompanying drawings.

### DESCRIPTION OF THE INVENTION

The best mode and the preferred embodiment of the novel wound care dressing is shown generally in FIGS. 1 through 4.

FIG. 1 illustrates a wound care dressing or sponge 10 formed with an inner porous polyurethane sponge or foam material body 12 with a substantially planar top and bottom surface having a thickness ranging from about 1 mm to about 3 mm in thickness, preferably about 2 mm in thickness. The use of the terms foam or sponge to describe the body of the wound dressing is interchangeable. The wound dressing body is naturally soft and requires no hydration prior to application while sustaining bacteriostatic activity for up to 7 days. The dressing absorbs bacteria-laden exudate out of and away from the wound which facilitates healing and aids in patient comfort. Bacteriostatic dressings better manage bioburden, which helps the body's own immune system restore bacterial balance. An outer adhesive covering 14 of film such as polyurethane, polyester, hydrogel, and hydrocolloids can be used. The repositionable cover adhesive is preferably a hydrogel adhesive but alternately can be a hydrocolloid adhesive and/or an acrylic adhesive. The sponge body 12 can bind up to 0.0010 g/g of each dye with the present dye take up of the commercial polyurethane material being used less than 0.0005 g/g. In the preferred embodiment, the polyurethane material of the body 12 is about 99.3% by weight. A gram-positive Methylene Blue dye of about 0.00035 g/g weight or less is bonded to the sponge polyurethane material and a gram-negative Crystal Violet dye of about 0.00035 g/g weight or less is bonded to the sponge polyurethane material. The dye will not leach from the sponge under normal circumstances and the leachable levels of the dyes are below 0.5 ppm.

Alternatively, other bactericidal dyes such as gram negative Dimethyl Methylene Blue (blue color); gram negative New Methylene Blue (blue color); gram positive Brilliant Green (green color); gram positive Malachite (green color); gram positive Acriflavine (orange color) and gram positive Quinacrine (yellow color) may be added in the same gram weight noted above, namely 0.00035 g/g or less as long as one dye is gram positive and the other dye is gram negative so that the full spectrum of bacteria and other microbial entities can be acted upon. It has been found that when the aforementioned dyes are added to the polyurethane foam at a concentration ranging from 0.0001 to 0.00035 g/g weight provides a complete biocidal effect throughout the bacterial spectrum and the combination of gram positive and gram negative dyes respectively act on the full spectrum of bacteria.

While the action of the dyes on bacteria is not fully known it is believed that when the dyes come in contact with proteins within bacteria cells they detach from their polymer binding site and enter the cell. Inside the cell it is believed that the dyes may interrupt the electrical pathway of the cell preventing it from reproducing. In short it is theorized that the dyes operate by “short circuiting” electron transport pathways. Various dyes show differential activity towards gram-negative versus gram-positive bacteria and also bind differently to different polymers.

No oxidizing dyes or oxidizing materials such as silver oxide should be added to the foam material as the shelf life and biocidal effectiveness of the foam material will be compromised.

The outer adhesive used on the cover and border 14 around the foam body 12 can be made of medical grade adhesive preferably hydrogel. The body 12 of the polyurethane foam ranges from 1 mm to 10 mm in thickness, preferably 5 mm.

Polyurethane has been selected because of its ability to be sterilized by gamma irradiation or electron beam without damage to the dressing, its absorbability of fluids, the ability to be treated with microbial materials and because it can absorb shock through the flexible cell structure of the material while retaining shape when placed over a wound. The material is also soft and does not have to be hydrated. The wound care sponge 10 center section of polyurethane wicks fluid from the body wound while the cover and border 14 prevents tissue growth and the adhesive allows ease of repositioning of the wound dressing from the patient.

The base polyurethane material is commercially obtained from RYNEL®, Inc., a manufacturer of polyurethane foam and is designated as 562-B foam. As shown in FIG. 4, in step S1 the sheet of commercial polyurethane material is initially washed with a de-ionized water carrier preferably containing a sodium bicarbonate in the first washing cycle of 5 minutes at a temperature of ranging from approximately 40° to 100° F. and the washing is repeated in de-ionized water not less than two 5 minute cycles to remove surfactants and wetting agents. In step S2, the polyurethane foam is prepped with an alcohol solution ranging from about 2% isopropyl alcohol (IPA) to about 30% isopropyl alcohol (IPA) for a period of at least 5 minutes but not more than one hour to open the maximum polyurethane binding sites to accept the dyes. In addition to IPA, other miscible alcohols such as ethyl alcohol may be used. Once the polyurethane material has been prepped, the two dyes (Methylene Blue: Crystal Violet) are injected into the dye bath and the sponge is allowed to soak for a period of at least 2 minutes and not more than 1 hour, as shown in step S3. In step S4, the foam material is then continuously palpated while the dye bath is agitated. Preferably the bath is mechanically agitated at 3 cycles per minute; however the bath can be sonically agitated. The temperature of the bath ranges from about 40° to about 100° F. during the agitation. In step S5, the foam material complexed with the dyes is then removed from the bath and rinsed with de-ionized water a plurality of times until the rinse water washes away any unbound dye and is essentially clear to the eye washing away any unbound dye. Preferably three wash cycles are performed with sodium bicarbonate being used in the first cycle. In step S6, a surfactant BASF68, TWEEN 20 and glycerin are added to de-ionized water and the polyurethane sheet is soaked for 15 minutes so that the sheet is hydrophilic. In a final step S7, the foam sheet is then removed and any excess liquid is wrung out of the foam body using a standard mechanical spin or centrifuge at about 500 to about 1000 rpm. The foam can bind up to 0.0010 g/g of each dye with the present take up of the preferred embodiment of polyurethane material being manufactured being less than 0.0005 g/g for each dye preferably ranging from about 0.00015 g/g to about 0.00035 g/g. The sponge is dried at about 100° F. for 2 hours using fan-blown purified air.

The polyurethane sponge body used is a white open-celled sponge, with instantaneous fluid wicking. The formed sheet of polyurethane material is preferably about 5 mm but falls within the range of 1 mm to 10 mm in thickness and the sheet can be cut in a sponge pad length of 6 to 10 inches, with a preferred length of 8 inches and a width of about 2.5 inches to 3 inches preferably 2.75 inches. A 1 mm to 3 mm thickness can be used when it is desired to view the color change on the side opposite the wound site. A plastic film covered border 14 having a hydrogel adhesive is secured to the sponge pad which was previously cut from the processed sheet to a desired size.

Any of a variety of additional substances can be later introduced into the finalized polyurethane dressing washing by soaking or immersing the polyurethane dressing in a solution of the desired substance(s) followed by drying of the polyurethane. Substances which can be readily incorporated in the polyurethane sponge by this or any other suitable manner include antimicrobials and/or antibiotics such as erythromycin, bacitracin, neomycin, penicillin, polymyxin B, tetracycline viomycin, chloromycetin and streptomycins, cefazolin, ampicillin, tobramycin, clindamycine and gentamycin, etc.; amino acids, peptides, vitamins, co-factors for protein synthesis; hormones; synthesizers; enzymes such as collagenase, peptidases, oxidases, etc.; angiogenic drugs and polymeric carriers containing such drugs; biocompatible surface active agents; antigenic agents.

The polyurethane sponge wound dressing when treated with the Methylene Blue and Crystal Violet has been found to be effective against the bacteria MRSA, Staphylococcus aureus, Staphylococcus epidermidis, Pseudomonas aeruginosa, Pseudomonas florescens, Escherichia coli, Enterococcus faecalis, VRE, Streptoccoccus pyogenes, Klesbsiella pneumoniae. Proteus mirabilis, Proteus vulgaris, and Enterobacter aerogenes, Bacillus subtilis, Yersinia enterocolitica, Serratia marcescens, Streptococcus pneumoniae, and Yeast, Candida albicans, Candida krusei, and Candida glabrata. The killing activity continues in the dressing until the dyes are used up in the sponge and it turns white. The color of the body 12 of the wound dressing is originally blue and infection at the wound site turns the dressing white. Thus color change in the sponge can act as an indicator when the sponge is lifted to determine if a wound is infected or not. The sponge structure can be sterilized with gamma irradiation or electron beam without degradation of the dressing structure and compromising the dye bactericidal effects.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What is claimed is:

1. A wound dressing comprising a body made of sterilized polyurethane foam material treated with a plurality of colored biocidal dyes having non-oxidant gram-positive and gram-negative characteristics to bind the dyes to binding sites in the polyurethane, said dyes imparting in the wound dressing body a distinct color, said body ranging from about 1 mm to about 10 mm in thickness and having an repositional adhesive cover and border secured to said body, said body when placed in contact with an infected wound site changing color.

2. A wound dressing as claimed in claim 1 wherein said wound dressing has been sterilized with gamma rays.

3. A wound dressing as claimed in claim 1 wherein said foam body is originally colored blue and has a color change to white when placed on an infectious wound site.

4. A wound dressing as claimed in claim 1 wherein said wound dressing body has a composition by weight of more than 99% polyurethane material containing about 0.00010 g/g to about 0.00035 g/g weight a germicidal gram-positive dye and about 0.00010 g/g to about 0.00035 g/g weight a germicidal gram positive dye.

5. A wound dressing as claimed in claim 1 wherein said wound dressing body has a composition by weight of about 99.3% polyurethane material, about 0.00015 g/g to about 0.00035 g/g weight Methylene Blue dye and about 0.00015 g/g to about 0.00035 g/g by weight Crystal Violet dye.

6. A wound dressing as claimed in claim 1 wherein said wound dressing body has a cover of plastic film with a hydrogel adhesive.

7. A wound dressing as claimed in claim 1 wherein said cover and border is a plastic film taken from a group consisting of polyurethane, polyester and hydrogel.

8. A wound dressing as claimed in claim 1 wherein said polyurethane wound dressing ranges from about 1 to about 3 mm in thickness.

9. A wound dressing as claimed in claim 1 wherein said polyurethane wound dressing is about 5 mm in thickness.

10. A sterilized wound dressing comprising a body section made of polyurethane foam material treated to open binding sites to a plurality of colored biocidal dyes with at least one of said dyes being gram positive and at least one of the said dyes being gram negative bonded to said polyurethane foam material, said dyes coloring the body section a distinct color, said body section ranging from about 1 mm to about 10 mm in thickness and a plastic film cover and border with a hydrogel adhesive secured to said cover and border providing means to reposition said wound dressing on a patient, said body section when placed in contact with an infected wound site changing color indicating an infection.

11. A wound dressing as claimed in claim 10 wherein said wound dressing body has a composition by weight of over 99.3% polyurethane material, about 0.00015 g/g to about 0.00035 g/g weight Methylene Blue dye and about 0.00015 g/g to about 0.00035 g/g weight Crystal Violet dye.

12. A wound dressing as claimed in claim 10 wherein said wound dressing dye weights are greater than 0.00025.

13. A method of constructing a color change wound dressing comprising the steps of:
a). washing a polyurethane foam sheet to eliminate wetting agents;
b). prepping the foam sheet with an alcohol solution;
c). applying a plurality of colored biocidal dyes to the prepped foam sheet, for a suitable period of time to bind said dyes to the polyurethane foam at a concentration ranging from at least 0.00025 grams per gram to not more than 0.0005 grams per gram of dye for every gram of foam;
d). washing the dyed polyurethane foam sheet to remove unbound dye;
e). adding wetting agents to the polyurethane foam sheet; and
f). removing excess liquid from the foam sheet and drying the polyurethane dyed foam.

14. A method as claimed in claim 13 wherein in step d) said plurality of colored germicidal dyes applied to said polyurethane foam sheet, contain at least one dye which is gram positive and at least one dye which is gram-negative.

15. A method as claimed in claim 13 wherein said colored biocidal dyes are Methylene Blue dye and Crystal Violet dye.

16. A method as claimed in claim 13 wherein in said step f) removing excess liquid from the foam comprises spinning said sponge at about 500 to about 1000 rpm.

17. A method as claimed in claim 13 wherein step e) further comprises washing the dye polyurethane foam a plurality of times until the last wash is clear.

18. A method as claimed in claim 13 wherein in step c) prepping includes treating the polyurethane foam with an alcohol solution ranging from about 2% to about 30% alcohol for a period of at least 5 minutes but not more than one hour.

19. A method of constructing a color changing wound dressing comprising the steps of:
a). washing a formed polyurethane foam sheet to eliminate foaming agents;
b). prepping the foam sheet with an alcohol solution for at least 5 minutes;
c). applying a plurality of colored biocidal dyes to the prepped foam sheet, for a suitable period of time to bind said dyes to the polyurethane foam at a concentration ranging from at least about 0.00015 g/g to about 0.00035 g/g;
d). washing the dyed polyurethane foam sheet a plurality of times until the wash water is clear; and
e). removing excess liquid from the foam sheet using a mechanical spin and drying the polyurethane dyed foam sheet;
f). cutting the foam sheet into sized sections; and
g). applying an adhesive covering to each sized section.

20. A method as claimed in claim 19 including a step after step of adding at least one wetting agent to the dyed polyurethane foam sheet.
